(19) United States
(12) Patent Application Publication
(10) Pub. No.: US 2010/0009856 A1
(43) Pub. Date: Jan. 14, 2010

(54) Randomized dna libraries and double-stranded rna libraries, use and method of production thereof

(75) Inventors: Zicai Liang, Sundbyberg (SE); Hong-Yan Zhang, Sundbyberg (SE); Meihong Cheng, Beijing (CN); Yan Shen, Beijing (CN)

Correspondence Address: KRATZ, QUINTOS & HANSON, LLP, 1420 K Street, N.W., Suite 400, WASHINGTON, DC, 20005, US

(73) Assignee: Sinogenomax Sompany LTD., Bejing (CN)

(21) Appl. No.: 10/517,324

(22) PCT Filed: Jun. 23, 2003
(86) PCT No.: PCT/SE2003/001077

§ 371 Date: Dec. 20, 2004

Related U.S. Application Data
(60) Provisional application No. 60/390,108, filed on Jun. 21, 2002.

(51) Int. Cl. C40B 30/00; C40B 40/08; C07H 21/04; C07H 21/02

(52) U.S. Cl. 506/7; 506/17; 536/23.1; 536/24.1

(57) ABSTRACT

This invention relates to DNA libraries based on plasmid or viral vectors that can express double-stranded RNA of 10-30 base pairs in length with all possible sequences, where each of the double stranded RNA is formed by a single RNA molecule in the form of hairpin, or formed by two separate RNA molecules with different 3′-overhangs. Each single member in such a DNA library encodes all components of a double stranded RNA as specified above. Such a library can be used in screening for double stranded RNA species that can induce a given phenotype without prior knowledge of their target genes. This invention further relates to a method to generate such a DNA library.

### TECHNICAL FIELD

This invention relates to DNA libraries based on plasmid or viral vectors that can express double-stranded RNA of 10-30 base pairs in length with all possible sequences, where each of the double stranded RNA is formed by a single RNA molecule in the form of hairpin, or formed by two separate RNA molecules with different 3′-overhangs. Each single member in such a DNA library encodes all components of a double stranded RNA as specified above. Such a library can be used in screening for double stranded RNA species that can induce a given phenotype without prior knowledge of their target genes. This invention further relates to a method to generate such a DNA library.

### BACKGROUND ART

Messenger RNA (mRNA) is normally perceived as the information-carrying intermediate in protein synthesis that is transcribed by RNA polymerase from a DNA template and subsequently translated by ribosome to generate protein molecules. Recently more data have demonstrated that many genes are transcribed into RNA molecules that are not translated into proteins at all (Okazaki Y et. AL., Nature; 420 (6915): 563-573 (2002)). SOME OF THE UNTRANSLATED RNA WERE FOUND TO CARRY OUT functions in the regulation of the other MRNA by inducing the degradation of the MRNA IN A SEQUENCE SPECIFIC MANNER (AMBROS V., CELL; 113 (6): 673-676 (2003)). THIS is in good agreement with the recent finding that double stranded RNA and synthetic siRNA can also induce cognate MRNA degradation in a wide range of organisms (MCMANUS M T, SHARP P A., NATURE REV GENET.; 3 (10): 737-747 (2002)). LONG double stranded RNA was found to induce intensive non-specific inhibition of RNA synthesis in mammalian cells, but siRNA can bypass this obstacle and still maintain the strong inhibitory effect on target gene which shares sequence identity with THE SIRNA (ELBASHIR S M ET AL., NATURE; 411 (6836): 494-498 (2001)). THIS HAS MADE siRNA a primary tool for gene knockdown in functional genomics. SIRNA also has the potential to become drugs that can be used to cure a disease by reducing the activity of disease related gene.

SIRNA are generally double stranded RNA of 19-25 base pairs that are either formed by a single RNA molecule in the form of hairpin or formed by two separate RNA molecules, with different 3′-overhangs. SIRNA can be produced in three ways: chemical synthesis; expression from DNA vectors under the drive of a promoter; and RNase III (Dicer) cleavage of long double stranded RNA. All siRNA that have been used so far are designed to target a segment of a predefined gene.

### SUMMARY OF INVENTION

The present invention relates to DNA libraries, each of which contains all possible permutations (permutation refers to different sequences) of double-stranded RNA of certain length. Such DNA libraries can be easily configured to produce all permutations of siRNA. It provides a high throughput screening method for double stranded RNA (as well as siRNA) in a target-independent manner for indications related to any given phenotype. More specifically, the siRNA encoded by such libraries can be used in such screening either individually, or as a mixture of any complexity, without the burden of knowing its sequence or its target gene. This method can overcome two major obstacles in siRNA application: 1) the incomplete knowledge about the transcriptome of each organism. According to the recent data from mouse transcriptome analysis, our knowledge about the transcriptome of this best understood model animal is still far from complete. Much less is known about the transcriptome of human and other animals. Since the application of our library does not require any prior information about the target sequence, it will allow immediate implementation of genome-wide siRNA screening in any organisms. 2) the extraordinarily high cost of siRNA. No matter how the siRNA is prepared, the cost of making siRNA targeting all known MRNA of an organism is extremely high. A single regenerateable DNA library that contains all permutation of siRNA that can be applied in any organisms virtually reduces the cost of siRNA production to a minimum level.

Accordingly, in one aspect the present invention relates to a DNA library for the production of a library of double stranded RNA molecules of a predefined length in the range of 10-30 base pairs in living cells, wherein the sequence (s) of the DNA region (or regions) encoding the double stranded part of double stranded RNA molecule (s) is randomized in a number selected from 4 to all nucleotide positions, and wherein both strands of said double stranded RNA molecule is produced from a single member of the DNA library. The invention also provides a kit containing the DNA library.

In another aspect the present invention provides a method of preparing the DNA library.

In yet another aspect the invention relates to an RNA library obtained from the DNA library.

Further aspects and advantages of the invention will become evident hereinafter from the following detailed description and attached claims.

### DESCRIPTION OF THE DRAWINGS

FIG. 1 shows an example of construction of DNA library that can encode all permutations of double stranded RNA of a certain length. Example 1, a DNA library that can encode all double stranded RNA with 19 base pair duplex region and 3′ poly U over hangs. In FIG. 1A, the cloning strategy is shown. In FIG. 1B, experimental verification of the quality of the library is demonstrated. As shown in the agarose gel, single clone (1×), and pools of 10 clones (10×), and pools of 30 clones give rise to the a single expected band after enzyme cleavage, suggesting that most clones in the library contain the expected insert. The same procedure can be used to produce such DNA libraries encoding different length (10-30 base pair) of double stranded RNA, as well as such DNA libraries with only part of the DNA sequence (4-30 nt) randomized.

FIG. 2 shows the construction of a plasmid to verify that the presence of two promoters and two terminators in opposite sides of the RNA coding region can afford efficient down-regulation of the expression of the target gene. With all scientific knowledge available as of today, such an efficient down regulation can only be achieved by the efficient production of double stranded RNA from the plasmid. Thus it is concluded that this plasmid can efficiently produced double stranded RNA in living cells. A shows the cloning strategy. B shows the gel analysis verified that the designed fragment is inserted into the plasmid. C illustrates cell assay verified that the resulting plasmid induces efficient inhibition of target gene Renilla luciferase.

FIG. 3 shows an example of an alternative method of generating DNA libraries that encode all permutations of double stranded RNA of a given length. In FIG. A, the cloning strategy is shown. In FIG. B, sequences of the different segments in A with key restriction sites underlined are shown. The same procedure can be used to produce such DNA libraries encoding different length (10-30 base pair) of double stranded RNA, as well as such DNA libraries with only part of the DNA sequence (4-30 nt) randomized.

FIG. 4 shows another alternative method of generating DNA libraries that encode all permutations of double stranded RNA of a given length. A illustrates the cloning strategy. B illustrates sequences of the different segments in A with key restriction sites underlined. The same procedure can be used to produce such DNA libraries encoding different length (10-30 base pair) of double stranded RNA, as well as such DNA libraries with only part of the DNA sequence (4-30 nt) randomized.

### DETAILED DESCRIPTION OF THE INVENTION

Small interference RNA (siRNA) is a term initially used to define short double stranded RNA that have a 19-21 nt double-stranded region nested between 3′-UU or TT or other single stranded overhangs. A number of variations of this original form of siRNA (such as hairpin-type) have been introduced lately. Such siRNA can be used to reduce the expression of genes having identical sequence to the siRNA double stranded region in cells from a variety of different organisms. While longer double stranded DNA and RNA also could be produced by means of the methods of the invention, the libraries of the invention have been restricted to double stranded DNA and RNA of a length of 10-30 base pairs, since above the length of 30 base pairs, the nucleotides will be more likely to produce an immunoresponse, and other disturbing side-effects when transfected into living cells.

SIRNA are initially chemically synthesized, but several methods have been introduced to generate siRNA enzymatically, using viral promoters such as t7 promoter, or microRNA promoter such as H 1 or U6, in free form or in plasmid or viral vectors.

The current invention provides a method to construct DNA libraries encoding random siRNA libraries. Such a library differs from the prior art in that in the prior art, one would have to design the siRNA according to a known sequence of the gene, whereas from the present library one can screen through a fully random panel of different siRNA (without the need of prior knowledge of their sequences or their target sequences) to look for phenotypes associated with each siRNA, and then identify the genes related to each siRNA de novo.

### Construction of DNA Libraries Containing a Single Randomized Region

The challenge of making a fully randomized DNA library based on plasmids or viral vectors encoding all permutations of siRNA is to make sure that each member of the DNA library expresses a distinct and complete double stranded RNA. None of the existing methods of making vector-based siRNA (short double stranded RNA) can meet this challenge.

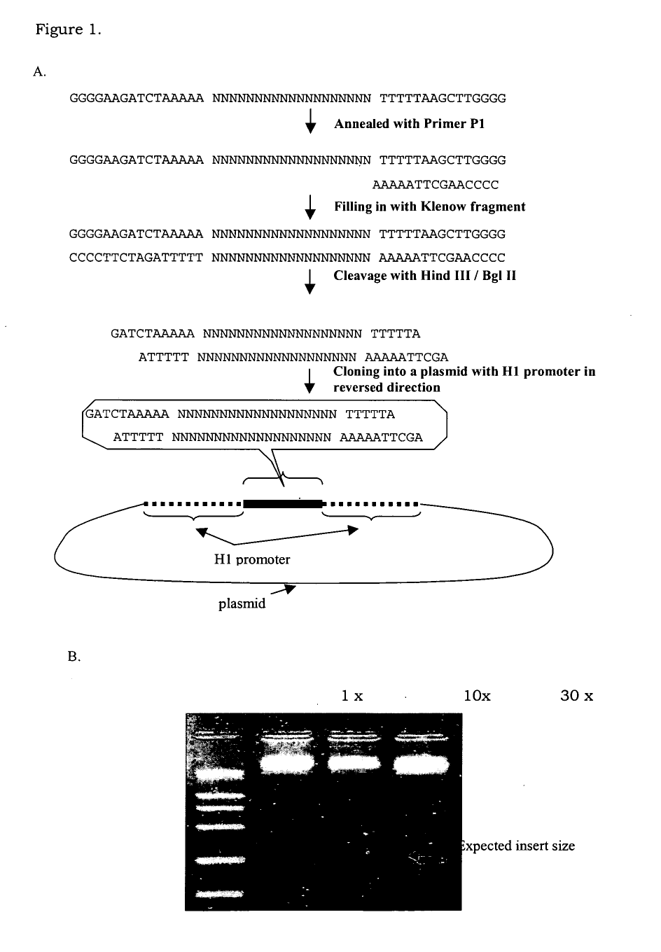

The current invention describes the construction of a random DNA library with only one randomized region. Then for each plasmid, two promoters will drive the transcription of this region from the opposite direction to produce the two complementary RNA strands separately. Two transcription terminators were placed at each end of the randomized region to make sure that RNA of a defined length can be produced from each direction. The advantage of this approach is to avoid the troublesome cloning procedure in the dual-region system as will be described beneath for creating two reverse complementing regions in each individual plasmid. One example of the promoters that can be used in such a system is the RNA polymerase III promoters H1 or U6. For RNA polymerase III, a stretch OF TTTTT is needed for the proper termination of the transcription. In order to use this RNA polymerase to drive expression of the same region from both directions, the TTTTT stretch has to be inserted on the both ends of the randomized region. There is one problem though: the RNA polymerase III promoters has to be placed immediately next to the randomized region to ensure proper transcription start from the precise location of the beginning of the randomized region, but those promoters does not contain a AAAAA stretch that would allow THE TTTTT terminator to appear on the opposite direction. The only way this can be done is to mutate the RNA polymerase III promoters to insert such a AAAAA stretch, and nobody knows how the insertion of the AAAAA stretch will affect the transcription starting, and the rate of transcription. As will be shown below, we mutated the HI RNA polymerase III promoter and inserted an AAAAA stretch at the end of the promoter and verified that the mutated promoter support proper transcription start and product of effective siRNA. Thus, we first started to construct a plasmid library with the termination signal placed on both sides of the randomized region (FIG. 1).

Construction of the Vector with Dual-H1 Promoter Against Renilla Luciferase

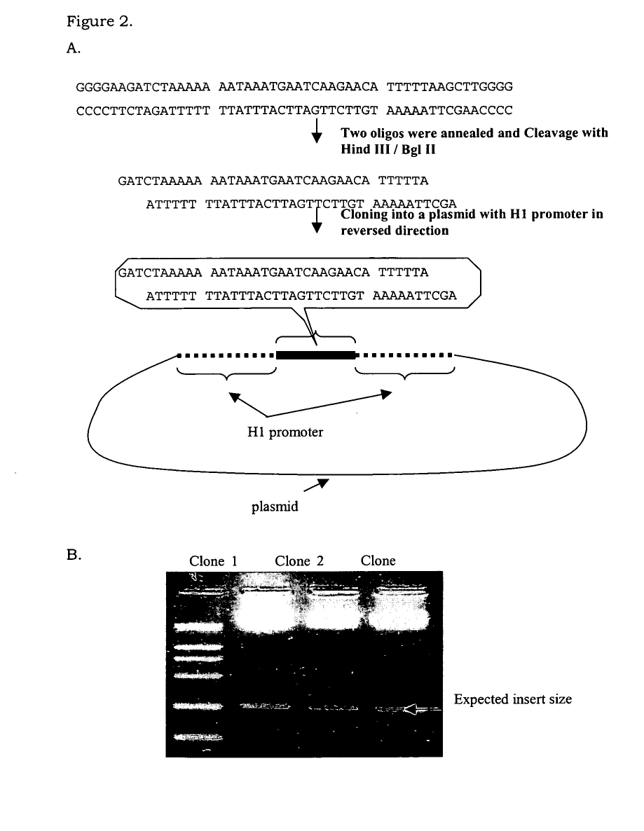

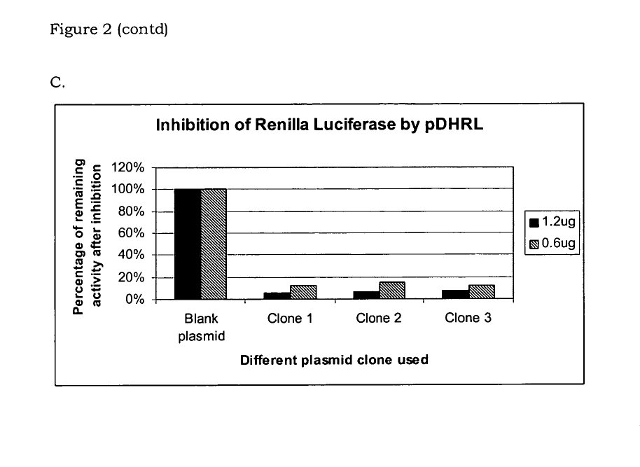

A plasmid with two mutated RNA polymerase III promoters, each embedding one transcription terminator sequence for the other promoter, was constructed with the siRNA region designed to target a model molecule Renilla luciferase (FIG. 2). The key finding that such a plasmid can support the successful production of effective siRNA duplex from a single target sequence of 19 bp forms the basis of constructing fully randomized siRNA library that have only one randomized region (FIG. 2).

Mutation of the H 1 RNA polymerase III promoters and construction of the example plasmid is described in details below.
1. Delete 3 nucleotides immediately upstream of Bgl II site in pBLUESCRIPT II KS-H1 vector (Brummelkamp T R et al., Science, 296 (5567): 550-553 (2002)) PCR amplify the fragment between EcoR I-BGL II (H 1 promoter) of the original vector, with the following primers:

5′primer: GGAATTCGAACGCTGACGTCATCAACCCG

3′ primer: GAAGATCTGTCTCATACAGAACTTATAAGATTCCC (matation one: three (3) nucleotides just upstream of Bgl II site was deleted in order for transcription to start from proper position after the insertion of the AAAAA sequence according to described beneath) Clone the PCR product in between EcoR I-BGL II, into the original pBLUESCRIPT II KS-H 1 (Brummelkamp T R et al. cited above) vector, verify the plasmid DNA by sequencing:
The modified sequence:

| | |
|---|---|
| 001 | TCCAGGNANC GCGGGCCCAG TGTCACTAGG CGGGAACACC |
| | CAGCGCGCGT |
| 051 | GCGCCCTGGC AGGAAGATGG CTGTGAGGGA CAGGGGAGTG |
| | GCGCCCTGCA |
| 101 | ATATTTGCAT GTCGCTATGT GTTCTGGGAA ATCACCATAA |
| | ACGTGAAATG |
| 151 | TCTTTGGATT TGGGAATCTT ATAAGTTCTG TATGAGACAG |
| | ATCTTCAATA |
| 201 | TTGGCCATTA GCCATATTAT TCATTGGTTA TATAGCATAA |
| | ATCAATATTG |
| 251 | GCTATTGGCC ATTGCATACG TTGTATCTAT ATCATAATAT |
| | GTACATTTAT |
| 301 | ATTGGCTCAT GTCCAATATG ACCGCCATGT TGGCATTGAT |
| | TATTGACTAG |
| 351 | TTATTAATAG TAATCAATTA CGGGGTCATT AGTTCATAGC |
| | CCATTATGGG |
| 401 | AGTTCCGCGT TACATAACTT ACGGTAAATG GCCCGCCTGG |
| | CTGACCGCCC |
| 451 | AACGACCCCC GCCCATTGAC GTCAATAATG ACGTATGTTC |
| | CCATAGTAAC |

2. Construction of the vector with mutated DUAL-H 1 promoters (here below referred to as pDH, stands for plasmid with Dual H 1 promoters) PCR amplify the fragment between EcoR I-BGL II of the above modified vector, with the following primers:

5′primer: ACGCGTCGACGAATTCGAACGCTGACGTCATCAACCCG
3′primer: CCCAAGCTTGTCTCATACAGAACTTATAAGATTCCC

Clone the above PCR product in between Sal I-HIND III, in a reversed orientation, into the above modified vector, verify the plasmid DNA by Bgl II+SAL I digestion, the correct clone should have a FRAGMENT OF-1 000BP. Results showed that all the ten clones checked were correct ones (Please note: pDH actually contains two truncated H 1 promoter, this is due to the need to subsequent cloning process. The missing part of the promoter will be made up during the subsequent cloning process.)
3. Put the Renilla luciferase target sequence into pDH to form PDHRL: A sequence corresponding to nt 82-100 of Renilla luciferase MRNA was used as the test DNA. siRNA targeting this site of the Renilla luciferase was known to be active (Brummelkamp T R et al. cited above). Two oligo DNA were synthesized and annealed to each other to make the double-stranded DNA:

5′GGGGAAGATCTAAAAAAATAAATGAATCAAGAACATTTTTAAGCTTGG
GG;
5′CCCCAAGCTTAAAAATGTTCTTGATTCATTTATTTTTTTAGATCTTCC
CC

The above double stranded DNA was cleaved with Bgl II-Hind III and cloned in between Bgl II-Hind III sites in pDH. Verification of the correct insertion of the DNA fragment into the plasmid DNA was done by cleavage by Bgl II+SAL I digestion, where the correct clone should give rise to A-250BP fragment. All three clones tested showed to have the correct insert (FIG. 2)

Efficient inhibition of luciferase expression by PDHRL. Take the above three clones: clone 1, clone 2 and clone 3, and transfect plasmid into HEK293 cells on 24-well plate, at 1.2 ug, 0.6 ug respectively, together with plasmid of Renilla luciferase and firefly luciferase encoding plasmids. 48 hours later, measure the Renilla and Firefly Luciferase activity. (FIG. 2C). The results suggested that with the mutated promoters the plasmid can induce very efficient inhibition of the expression of target gene Renilla luciferase, which indicated efficient production of siRNA from the mutated H 1 promoters in the dual promoter/dual terminator plasmid constructed in the current invention. Specifically the result suggested that with the mutated H 1 promoter, the RNA transcription driven by RNA polymerase III can be properly initiated and terminated, to result in the efficient production of duplex RNA of proper length that can induce significant RNA interference and inhibition of gene expression.

Cloning the Randomized DNA into pDH to Form a Library that Encodes all Permutations of the siRNA

The construction of randomized DNA library that encodes all permutations of siRNA is done in a similar way as the construction of the anti-luciferase siRNA encoding plasmid in PDHRL, with the only difference that the second strand of the tester sequence was generated ENZYMATICALLY to preserve the randomized nature of the sequence.

Three oligonucleotides were synthesized with 19, 20 and 21 nt of randomized region embedded within the two known sequences.

19-mer randomized region
GGGGAAGATCTAAAAANNNNNNNNNNNNNNNNNNN TTTTTAAGCTTGGG
G
20-mer randomized region
GGGGAAGATCTAAAAA NNNNNNNNNNNNNNNNNNNNTTTTTAAGCTTGG
GG
21-MER randomized region
GGGGAAGATCTAAAAA NNNNNNNNNNNNNNNNNNNNN TTTTTAAGCTT
GGGG

The oligonucleotides were allowed to anneal to a primer CCCCAAGCTTAAAAA and filled in with Klenow fragment in the presence of 1 mM concentration of dNTP in proper buffer (all chemicals other than DNA oligonucleotides were purchased from New England Biolabs Inc. unless otherwise specified). The duplex oligos were cleaved with Bgl II-Hind III and cloned in the Bgl II-Hind III sites of the pDH to form PDH-LIBRARYA.

The quality of the pDH-libraryA was assessed by first clone length analysis of 41 clones, where single clone, a 10-clone pool and a 30-clone pool was used to prepared plasmid DNA and cleavage with restriction enzyme. The results suggested that all clones have the insert of the same length (FIG. 1B). The ten clones were individually prepared and sequenced. All sequenced clones contain the expected 19 base pair insert as expected. Their sequences showed expected randomness as well (see below).

AAAGGGTTTACGTGGTTGG AATCGTCTTATTTGCATGC
AATTGACATGTGAGCTTGG AGTAGCTTGTTGAGGTTGG
CAGCATCACTGTATGTGTC CTATCTTCGTGGAGGTTGG
CTATGAAGGTGGTGATGCG CTTAATTGGTGGTTGTAGG
TGGCTGTATGTGAGTGGCT TTAATCTCTGGTGTCCTAA
TTGTAGGGACTTGGATGAT

One alternative to plasmid vectors for epitopic expression of foreign gene is various types of viral vectors. Since all cloning strategies for constructing viral vectors are common knowledge, and anybody with reasonable knowledge of the art can produce viral constructs that can carry out similar expression functions as the plasmids, the disclosure of making DNA libraries as above will also enable the production of DNA libraries as such in viral vectors.

Construction of DNA Libraries Containing a Pair of Randomized Regions with Inverted Sequences

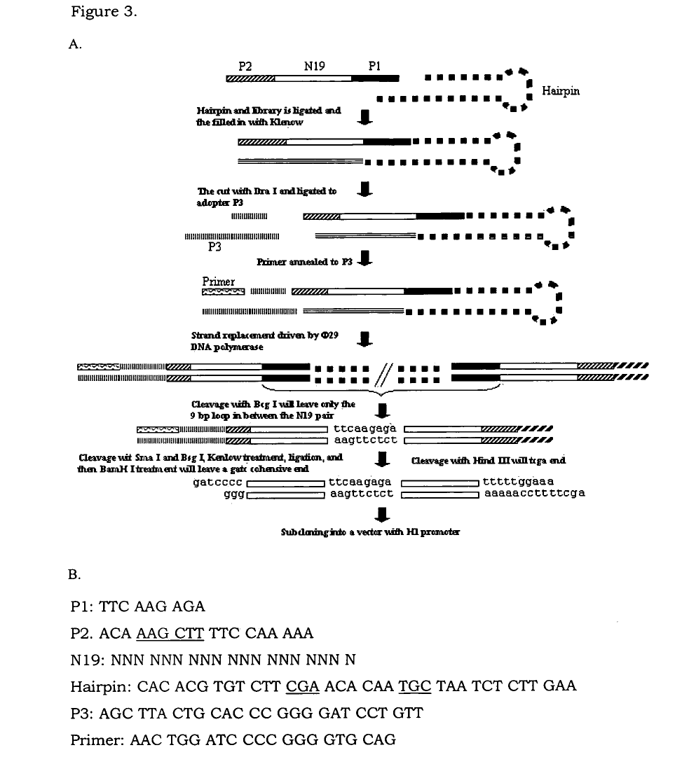

Although the vectors with two promoters and two terminators as represented by PDHRL and PDH-LIBRARYA are the preferred modes of the current invention, other methods of forming DNA libraries that encode all permutations of siRNA become obvious once the concept of DNA library encoding all permutations of siRNA is disclosed here. One such method is to form a plasmid library that encodes all permutations of the hairpin form of the siRNA. As an example, such a library can be formed according to the following procedure.
1. Library oligonucleotide was synthesized to contain a fully randomized region of 19 nt randomized sequence nested between two predetermined sequences (P1 and P2) with 5′ end phosphorylated. A hairpin forming oligonucleotide was synthesized to contain 5′ phosphorylation and a 3′ protruding stretch with complementary sequence of the P1 region. The Library oligonucleotide and the hairpin DNA were annealed and ligated with T4 DNA ligase and then filled in with Klenow fragment (FIG. 3)
2. The extension mixture, after purification, was cleaved with BamH I and ligated into an double stranded adopter that has cohesive ends on one end and a 3′ protruding stretch as a site for further priming (P3) After ligation the DNA are size-selected so that only full length fragments that contain library oligonucleotides and the hairpin oligonucleotide, as well as the adopter linker are collected.
3. Purified full length fragments are allowed to anneal to the primer 3 (which is complementary to the P3 priming site), and a strand-displacing DNA polymerase Phage29 DNA polymerase is used to drive the synthesis of a DNA fragment ALPHA. Each DNA fragment ALPHA contains: a fully double stranded adaptor linker on each end of its sequence, two identical copies of a randomized sequence arranged in reverse orientation, and the two copies are linked by the linearized sequence of the hairpin linker in its double stranded form.
4. DNA fragment ALPHA can then be cleaved in proper sites in its adopter linker region and then ligated to a plasmid for further manipulation (Plasmid alpha).
5. Plasmid alpha is first cleaved with Sam I and Bpm I and the filled in with Klenow fragment and ligated. The resulting plasmid is propagated in E coli and then the insert is cleaved with Bcg I to remove extra sequences between the two randomized region and leave a 9-nt stretch (TTCAAGAGA) to form the loop in the future siRNA hairpin (FIG. 13).
6. Afterwards, the insert can be all cleaved from the plasmid with Hind III and Bgl II and inserted into the PBLUESCRIPT-HL vector to form a library. This library encodes all siRNA permutations in a hairpin form. In this case, the plasmid only need to have one promoter and one terminator for the formation of hairpin RNA within the cells.

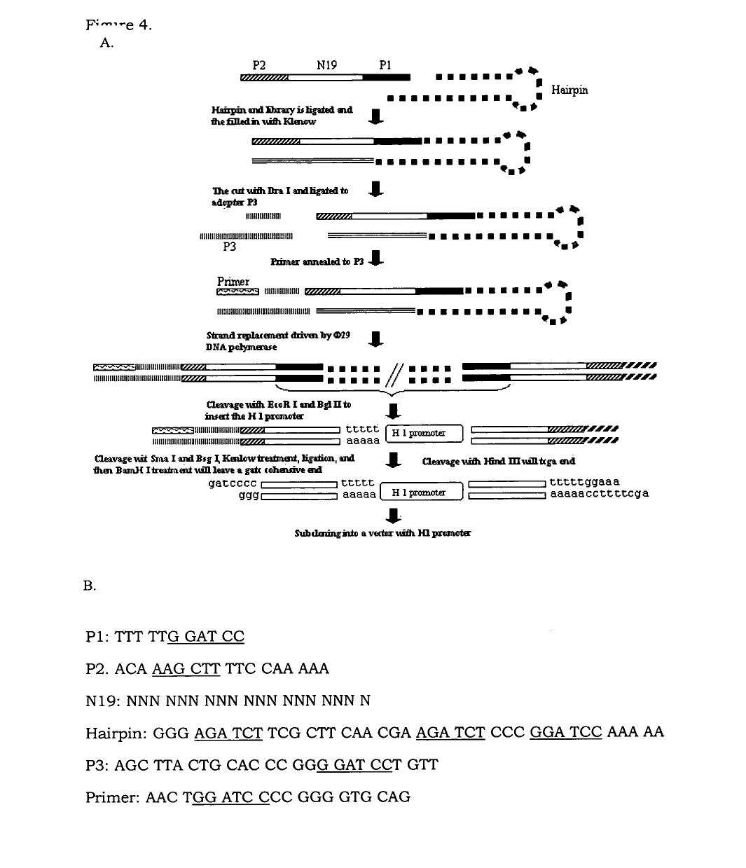

Slight modification of the above cloning protocol as illustrated in FIG. 4 can result in DNA libraries that have two wild type H 1 promoter and two transcription terminators, wherein each member of the library encode the two separate strands of a double stranded RNA. This involves the insertion of a second promoter AND TTTTT terminator between the two inverted randomized region of the DNA library as illustrated in FIG. 4. With the detailed disclosure described above and in FIG. 1-3, this alternative is obvious to a person skilled in the field.

It has to be stressed that due the enzymatic handling of the library, all siRNA that contain the restriction enzyme sites are lost. This will result in about 0.025% siRNA loss each restriction enzyme used. So in this sense the preferred mode of the current invention, based on two promoters and two terminators, will suffer less siRNA loss and be a more complete library, than the library generated according to the above hairpin library protocol due to the number of enzymes used in the individual protocols. Since the library contains about 2.75×1011 permutations in theory, the loss of siRNA species caused by the use of restriction enzymes will only have neglectable effect on the quality of library and for the screening of active siRNA against any specific gene. In the text of this invention, the referral to “all permutations of siRNA” should be understood as having this effect considered and included. Further elimination of this effect will be done by eliminating the use of restriction enzymes in the construction of the libraries.

Another note is that the sequences and restriction enzymes are only one set of examples that can be used to carry out the construction of the plasmid. The person skilled in the art can easily choose different restriction enzymes and corresponding sequences of the oligonucleotides to carry out the construction in similar manner in plasmids and viral vectors, according to the principle disclosed as above.

Generation of DNA Libraries that Encode Cell-Specific, Tissue-Specific or Species Specific Double Stranded RNA

With the disclosure of the random DNA libraries encoding all permutations of double stranded RNA of a given length, the method of establishing DNA libraries that encode cell-specific, tissue-specific or species specific double stranded RNA should be considered to be obvious to a person skilled in the field. One example of constructing such DNA libraries is presented below.

An oligonucleotide with 19 nt of randomized region is allowed to hybridize to MRNA purified from a specific cell type. The MRNA can be immobilized onto a streptavidin coated solid support (plastic beads for example) via biotin added to the end of the MRNA with Poly (A) polymerase. Immobilization of MRNA can be done in other ways too. After hybridization, all unbound DNA oligonucleotides are washed away and the bound DNA sub-random oligonucleotides are collected and cloned into the vector in a protocol identical to protocols described for fully randomized DNA oligonucleotides. The libraries resulted from this process will be highly enriched for molecules that encode double stranded RNA with sequence identical to the MRNA sources.

It should be noted that although all cloning procedures herein are described in the context of a single plasmid vector, the principle should be applicable to all types of plasmids, and the cassette containing the mutated promoters, terminators and the coding region of the DNA libraries can be transferred between those different types of plasmids.

It should be further noted that although all cloning procedures are described in the context of a single type of promoter, H1 promoter, the principle should be applicable to all types of RNA polymerase III type of promoters.

One alternative to plasmid vector for epitopic expression of foreign gene is various types of viral vectors. Since all cloning strategies for constructing viral vectors are common knowledge, and anybody with reasonable knowledge of the art can produce viral constructs that can carry out similar expression functions as the plasmids, the disclosure of making DNA libraries as above should also enable the production of DNA libraries as such in viral vectors.

### SUMMARY

The current invention involves DNA libraries that can generate double stranded RNA of 10-30 base pair in length, with at least one strand of the double stranded RNA having single stranded overhangs, and further involves methods to produce such DNA libraries. It is acknowledged that most frequently used double stranded RNA is siRNA of 19-21 base pair in length, normally with TT or UU overhangs on at least one of the strands. So the advantage of the current invention is discussed in comparison to siRNA generated by other methods.

In practice, only one in three to five or so short double stranded RNA that fulfill the basic structural requirement (19-21-base pair double stranded region, 3′ single stranded overhangs (normally TT, or UU, but not limited to such overhangs). For knocking down the 30,000 human genes using siRNA, about 90,000-150,000 siRNA then will have to be synthesized, at the cost of 18-30 million US dollars.

Similar amount of cost has to be allocated to any additional organism for which the full spectrum of siRNA will be generated for all genes The current invention can generate a siRNA library encoded in plasmids that contains in theory all the permutations (419=2.75×10¹¹) of siRNA (19 base pair duplexes plus overhangs) (the size of libraries for double stranded RNA of other length can be easily calculated in similar way), that can be used in any organisms for which the a proper promoter (s) can be found). The cost of generating this library is just a minimal fraction of the cost of synthesizing all siRNA chemically. In other words, this is a library with the complexity of 2.75×10¹¹ that contains reagents that can silence any gene in a mammalian and non-mammalian system. This is a very powerful toolbox for high throughput genome wide functional genomics and drug target screening, as well as nucleic acid drug development.

The complexity of this library can be further reduced dramatically by introducing a one-step oligoselection on the Library oligonucleotides. Such an approach will lead to the creation of gene-, cell/tissue-, or organism-specific siRNA encoding library that has much lower complexity (10²-10⁸), without sacrificing the usefulness of the library. Such a low complexity library can be partially or completely sequenced using different sequencing methods and enable the creation of plasmid collections that contains known siRNA encoders for each gene in an organism such as human, mouse or rat.

The description of the above is most based on plasmid system but the same library and collection can be easily established in viral vector using the same principle.

A few key classes of application of the invention is listed here as examples

1) A full collection of siRNA encoding plasmids can be selected for any given gene from this library through standard screening (which could be automated).
2) A full collection of siRNA encoding plasmids can be selected for any given cell type, tissue and organism can be established according to the invention.
3) Such collections of siRNA encoding plasmids can then be easily evaluated for their individual capacity to knockdown gene expression.
4) Most powerfully, such DNA libraries can be used for phenotype-based screening of target genes without prior knowledge of the target sequence or the siRNA sequences, thus the artisan can avoid the biased pre-selection of target genes. This will become one most significant way of functional annotation and drug target screening.

1: A DNA-library for production of a library of double stranded RNA-molecules (dsRNA) of a predefined length, the library consisting of double stranded DNA-molecules (dsDNA) where each dsDNA comprise a stretch wherein both strands contiguously encode a promoter, a dsRNA-encoding sequence of 10-30 base pairs encoding the dsRNA to be produced and a transcription termination sequence, wherein each of said promoters has been mutated to include the sequence complementary to the termination sequence of the other strand.

2: A DNA-library according to claim 1, wherein said promoters are H1-promoters or U6-promoters that have been mutated so as to incorporate an AAAAA-stretch at the end of the promoter, immediately next to the transcription starting site.

3: A DNA-library according to claim 1, wherein said dsRNA-encoding sequence is randomized in between 4 and all positions.

4: A DNA-library according to claim 1, wherein the produced dsRNA contains a single stranded region at one end.

5: A DNA-library according to claim 1, wherein the produced dsRNA contains single stranded regions at both ends.

6: A DNA-library according to claim 4, wherein at least one of the single stranded regions of the dsRNA is a poly-U overhang.

7: A DNA-library according to claim 4, wherein at least one of the single stranded regions of the dsRNA is a UU overhang.

8: A DNA-library according to claim 1, wherein it is constructed in a plasmid vector.

9: A DNA-library according to claim 1, wherein it is constructed in a viral vector.

10: A DNA-library according to claim 1, wherein the randomness of the library was modified by selection of the random DNA oligonucleotides, before cloning the said random DNA oligonucleotides into the vectors, through hybridization to a total RNA preparation or total mRNA preparation from a source, whereby only the oligonucleotides hybridized to the source RNA (or mRNA) are subsequently cloned into the vector, and wherein the source can be a cell, a cell line, a tissue, or a organism.

11: A kit containing the DNA-library according to claim 1.

12: An RNA-library obtained from the DNA-library according to claim 1.

13: A method of using the DNA-libraries of claim 1, wherein the library is transiently or permanently introduced into cells as a mixture.

14: A method of screening for double stranded RNA with biological functions comprising the use of the DNA-library according to claim 1.

15: A method of screening for novel genes comprising the use of the DNA-library according to claim 1.

16: An individual DNA-member of the DNA-library according to claim 1.

17: An individual RNA-member of the RNA-library according to claim 12.

18: Use of a DNA-molecule comprising the DNA-sequence AAAAA(N)ₙTTTTT, wherein (N)ₙ is a randomized region of 19, 20 or 21 nucleotides, in the production of dsRNA-molecules.

19: An H1 RNA-polymerase III-promoter mutated to have and AAAAA-stretch at the end of the promoter immediately ahead of the transcription starting site.

20: A plasmid with two mutated RNA polymerase III promoters, each embedding one transcription termination sequence for the other promoter, and a siRNA-encoding region between the promoters.

21: Any polymerase III-promoter mutated to have an AAAAA-stretch at the end of the promoter immediately ahead of the transcription starting site.


SEQUENCE LISTING (45 sequences)

SEQ ID NO: 1 - DNA, 29 bp, Artificial Sequence
  5?rimer
ggaattcgaacgctgacgtcatcaacccg

SEQ ID NO: 2 - DNA, 35 bp, Artificial Sequence
  3?rimer
gaagatctgtctcatacagaacttataagattccc

SEQ ID NO: 3 - DNA, 500 bp, Artificial Sequence
  Modified sequence
tccaggnancgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccattatgggagttccgcgttacataacttacggtaaatggcccgcctggctgaccgcccaacgacccccgcccattgacgtcaataatgacgtatgttcccatagtaac

SEQ ID NO: 4 - DNA, 38 bp, Artificial Sequence
  5?rimer
acgcgtcgacgaattcgaacgctgacgtcatcaacccg

SEQ ID NO: 5 - DNA, 36 bp, Artificial Sequence
  3?rimer
cccaagcttgtctcatacagaacttataagattccc

SEQ ID NO: 6 - DNA, 50 bp, Artificial Sequence
  oligo DNA
ggggaagatctaaaaaaataaatgaatcaagaacatttttaagcttgggg

SEQ ID NO: 7 - DNA, 50 bp, Artificial Sequence
  oligo DNA
ccccaagcttaaaaatgttcttgattcatttatttttttagatcttcccc

SEQ ID NO: 8 - DNA, 50 bp, Artificial Sequence
  19 nucleotides randomized region
ggggaagatctaaaaannnnnnnnnnnnnnnnnnntttttaagcttgggg

SEQ ID NO: 9 - DNA, 51 bp, Artificial Sequence
  20 nucleotides randomized region
ggggaagatctaaaaannnnnnnnnnnnnnnnnnnntttttaagcttgggg

SEQ ID NO: 10 - DNA, 52 bp, Artificial Sequence
  21 nucleotides randomized region
ggggaagatctaaaaannnnnnnnnnnnnnnnnnnnntttttaagcttgggg

SEQ ID NO: 11 - DNA, 15 bp, Artificial Sequence
  primer
ccccaagcttaaaaa

SEQ ID NO: 12 - DNA, 19 bp, Artificial Sequence
  19 base pair clone
aaagggtttacgtggttgg

SEQ ID NO: 13 - DNA, 19 bp, Artificial Sequence
  19 base pair clone
aatcgtcttatttgcatgc

SEQ ID NO: 14 - DNA, 19 bp, Artificial Sequence
  19 base pair clone
aattgacatgtgagcttgg

SEQ ID NO: 15 - DNA, 19 bp, Artificial Sequence
  19 base pair clone
agtagcttgttgaggttgg

SEQ ID NO: 16 - DNA, 19 bp, Artificial Sequence
  19 base pair clone
cagcatcactgtatgtgtc

SEQ ID NO: 17 - DNA, 19 bp, Artificial Sequence
  19 base pair clone
ctatcttcgtggaggttgg

SEQ ID NO: 18 - DNA, 19 bp, Artificial Sequence
  19 base pair clone
ctatgaaggtggtgatgcg

SEQ ID NO: 19 - DNA, 19 bp, Artificial Sequence
  19 base pair clone
cttaattggtggttgtagg

SEQ ID NO: 20 - DNA, 19 bp, Artificial Sequence
  19 base pair clone
tggctgtatgtgagtggct

SEQ ID NO: 21 - DNA, 19 bp, Artificial Sequence
  19 base pair clone
ttaatctctggtgtcctaa

SEQ ID NO: 22 - DNA, 19 bp, Artificial Sequence
  19 base pair clone
ttgtagggacttggatgat

SEQ ID NO: 23 - DNA, 15 bp, Artificial Sequence
  Primer
aaaaattcgaacccc

SEQ ID NO: 24 - DNA, 50 bp, Artificial Sequence
  complemantary to SEQ ID NO 8
ccccttctagatttttnnnnnnnnnnnnnnnnnnnaaaaattcgaacccc

SEQ ID NO: 25 - DNA, 35 bp, Artificial Sequence
  Cleaved from SEQ ID NO 8
gatctaaaaannnnnnnnnnnnnnnnnnnttttta

SEQ ID NO: 26 - DNA, 35 bp, Artificial Sequence
  Cleaved from SEQ ID NO 24
atttttnnnnnnnnnnnnnnnnnnnaaaaattcga

SEQ ID NO: 27 - DNA, 50 bp, Artificial Sequence; identical to SEQ ID NO: 6
  DNA/RNA sequence

SEQ ID NO: 28 - DNA, 50 bp, Artificial Sequence
  complemantary to SEQ ID NO 27
ccccttctagatttttttatttacttagttcttgtaaaaattcgaacccc

SEQ ID NO: 29 - DNA, 35 bp, Artificial Sequence
  cleaved from SEQ ID NO 27
gatctaaaaaaataaatgaatcaagaacattttta

SEQ ID NO: 30 - DNA, 35 bp, Artificial Sequence
  cleaved from SEQ ID NO 28
atttttttatttacttagttcttgtaaaaattcga

SEQ ID NO: 31 - DNA, 9 bp, Artificial Sequence
  DNA/RNA sequence
ttcaagaga

SEQ ID NO: 32 - DNA, 9 bp, Artificial Sequence
  Complemantary to SEQ ID NO 31
aagttctct

SEQ ID NO: 33 - DNA, 18 bp, Artificial Sequence
  DNA/RNA sequence
acaaagcttttccaaaaa

SEQ ID NO: 34 - DNA, 19 bp, Artificial Sequence
  Randomized sequence.
nnnnnnnnnnnnnnnnnnn

SEQ ID NO: 35 - DNA, 36 bp, Artificial Sequence
  DNA/RNA hairpin
cacacgtgtcttcgaacacaatgctaatctcttgaa

SEQ ID NO: 36 - DNA, 26 bp, Artificial Sequence
  Adopter
agcttactgcacccggggatcctgtt

SEQ ID NO: 37 - DNA, 21 bp, Artificial Sequence
  Primer
aactggatccccggggtgcag

SEQ ID NO: 38 - DNA, 64 bp, Artificial Sequence
  DNA/RNA Sequence
gatccccnnnnnnnnnnnnnnnnnnnttcaagagannnnnnnnnnnnnnnnnnntttttggaaa

SEQ ID NO: 39 - DNA, 64 bp, Artificial Sequence
  DNA/RNA Sequence
gggnnnnnnnnnnnnnnnnnnnaagttctctnnnnnnnnnnnnnnnnnnnaaaaaccttttcga

SEQ ID NO: 40 - DNA, 11 bp, Artificial Sequence
  DNA/RNA sequence
tttttggatcc

SEQ ID NO: 41 - DNA, 41 bp, Artificial Sequence
  DNA/RNA Hairpin
gggagatcttcgcttcaacgaagatctcccggatccaaaaa

SEQ ID NO: 42 - DNA, 31 bp, Artificial Sequence
  DNA/RNA Sequence
gatccccnnnnnnnnnnnnnnnnnnnttttt

SEQ ID NO: 43 - DNA, 29 bp, Artificial Sequence
  DNA/RNA Sequence
nnnnnnnnnnnnnnnnnnntttttggaaa

SEQ ID NO: 44 - DNA, 27 bp, Artificial Sequence
  DNA/RNA Sequence
gggnnnnnnnnnnnnnnnnnnnaaaaa

SEQ ID NO: 45 - DNA, 33 bp, Artificial Sequence
  DNA/RNA Sequence
nnnnnnnnnnnnnnnnnnnaaaaaccttttcga